(19) United States
(12) Patent Application Publication
(10) Pub. No.: US 2025/0022613 A1
(43) Pub. Date: Jan. 16, 2025

(54) METHOD OF DIFFERENTIATING PATTERNS OF CARE

(71) Applicant: International Business Machines Corporation, Armonk, NY (US)

(72) Inventors: George Sirbu, Saline, MI (US); William Ogallo, Nairobi (KE); Girmaw Abebe Tadesse, Nairobi (KE); Feng Xin, Cary, NC (US); Ajay Ashok Deshpande, Pleasantville, NY (US); Charles Muchiri Wachira, Karatina (KE); Aisha Walcott, Nairobi (KE); Vibha Anand, Chestnut Hill, MA (US); Brandi Hodor, Alexandria, KY (US); Zainab Savliwala, Ann Arbor, MI (US); Isaiah Mulang' Onando, Nairobi (KE)

(21) Appl. No.: 18/352,390

(22) Filed: Jul. 14, 2023

(51) Int. Cl. G16H 50/70

(52) U.S. Cl. CPC G16H 50/70

(57) ABSTRACT

A computer-implemented method for differentiating patterns of care (DPoC) to detect anomalous subsets in any given population with a defined set of outcomes and features. The computer-implemented method includes detecting the anomalous subsets, ranking the anomalous subsets based on a score of each anomalous subset that is reflective of an anomaly thereof, specifying whether each of the anomalous subsets overlaps with another one of the anomalous subsets, whether each of the anomalous subsets is unique and whether each of the anomalous subsets is conditional and specifying as to whether the detecting of each of the anomalous subsets has a higher or lower outcome than expected.

### BACKGROUND

The present invention generally relates to patterns of care, and more specifically, to methods of differentiating patterns of care (DPoC).

Healthcare is the improvement of health via the prevention, diagnosis, treatment, amelioration or cure of disease, illness, injury and other physical and mental impairments in people. Healthcare is delivered by health professionals and allied health fields. Medicine, dentistry, pharmacy, midwifery, nursing, optometry, audiology, psychology, occupational therapy, physical therapy, athletic training and other health professions all constitute healthcare. It includes work done in providing primary care, secondary care and tertiary care as well as in public health.

### SUMMARY

Embodiments of the present invention are directed to a computer-implemented method for differentiating patterns of care (DPoC) to detect anomalous subsets in any given population with a defined set of outcomes and features. The computer-implemented method includes detecting the anomalous subsets, ranking the anomalous subsets based on a score of each anomalous subset that is reflective of an anomaly thereof, specifying whether each of the anomalous subsets overlaps with another one of the anomalous subsets, whether each of the anomalous subsets is unique and whether each of the anomalous subsets is conditional and specifying as to whether the detecting of each of the anomalous subsets has a higher or lower outcome than expected.

As a result of an execution of the computer-implemented method, a data driven unsupervised learning approach will be enabled to automatically detect, rank, summarize and visualize anomalous subgroups whereby actionable features and values can be emphasized through conditional scanning and iterations of the process.

In accordance with additional or alternative embodiments of the invention, the computer-implemented method is characterized in that input data preparation, setup configuration, algorithm executions and report generations are automated.

In accordance with additional or alternative embodiments of the invention, the input data includes binary or numerical outcome data and feature data with an unlimited number of features.

In accordance with additional or alternative embodiments of the invention, the computer-implemented method further includes a feature selection operation in which the features of the unlimited number of features are selected.

In accordance with additional or alternative embodiments of the invention, the computer-implemented method further includes a parameter choice operation in which parameters include regularization parameters, counts for bootstrap repetitions, randomization initializations, numbers of the anomalous subsets to be identified and anomaly detection directions.

In accordance with additional or alternative embodiments of the invention, the specifying of whether each of the anomalous subsets overlaps with another one of the anomalous subsets, whether each of the anomalous subsets is unique and whether each of the anomalous subsets is conditional includes setting the specifying to specify one of whether each of the anomalous subsets overlaps with another one of the anomalous subsets, whether each of the anomalous subsets is unique or whether each of the anomalous subsets is conditional.

In accordance with additional or alternative embodiments of the invention, the computer-implemented method further includes summarizing and visualizing results of the detecting, the ranking, the specifying of whether each of the anomalous subsets overlaps with another one of the anomalous subsets, whether each of the anomalous subsets is unique and whether each of the anomalous subsets is conditional and the specifying as to whether the detecting of each of the anomalous subsets has a higher or lower outcome than expected.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 is a schematic diagram of a computing environment for executing a computer-implemented method for differentiating patterns of care (DPoC) to detect anomalous subsets in any given population with a defined set of outcomes and features in accordance with one or more embodiments of the present invention;

FIG. 2 is a block diagram of components of a machine learning training and inference system according to one or more embodiments of the present invention;

FIG. 3 is a flow diagram illustrating a computer-implemented method for differentiating patterns of care (DPoC) to detect anomalous subsets in any given population with a defined set of outcomes and features in accordance with one or more embodiments of the present invention;

FIG. 4 is a flow diagram that visualizes the a computer-implemented method of FIG. 3 in accordance with one or more embodiments of the present invention;

FIG. 5 is a graphical depiction of an identifying and ranking the anomalousness of multiple potentially overlapping subsets in accordance with one or more embodiments of the present invention;

FIG. 6 is a graphical depiction of an identifying and ranking the anomalousness of mutually exclusive subsets in accordance with one or more embodiments of the present invention; and

FIGS. 7A and 7B are graphical depictions of methods of regularization to control complexity in accordance with one or more embodiments of the present invention.

The diagrams depicted herein are illustrative. There can be many variations to the diagram or the operations described therein without departing from the spirit of the invention. For instance, the actions can be performed in a differing order or actions can be added, deleted or modified. Also, the term “coupled” and variations thereof describes having a communications path between two elements and does not imply a direct connection between the elements with no intervening elements/connections between them. All of these variations are considered a part of the specification.

In the accompanying figures and following detailed description of the disclosed embodiments, the various elements illustrated in the figures are provided with two or three digit reference numbers. With minor exceptions, the leftmost digit(s) of each reference number correspond to the figure in which its element is first illustrated.

### DETAILED DESCRIPTION

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

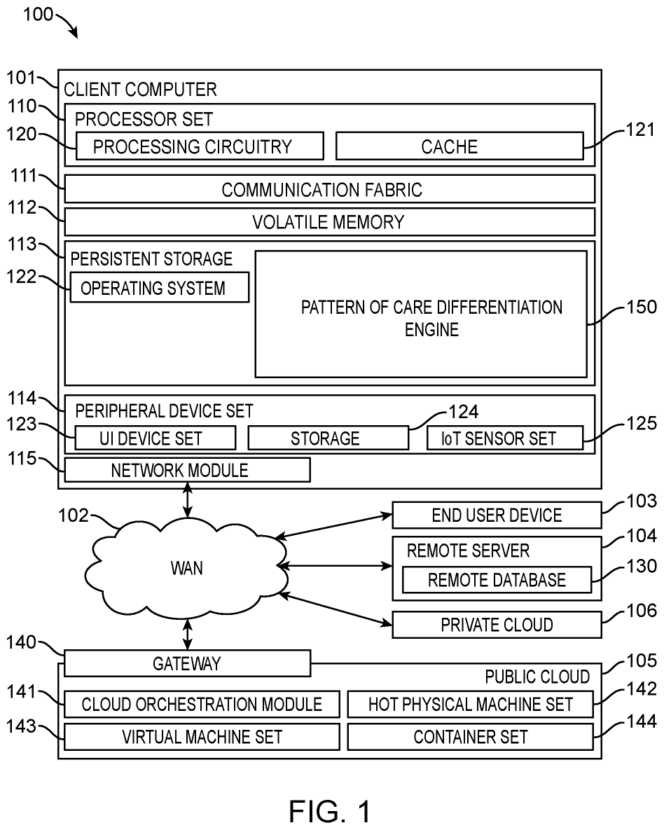

With reference to FIG. 1, a computer or computing device 100 that implements a computer-implemented method for differentiating patterns of care (DPOC) to detect anomalous subsets in any given population with a defined set of outcomes and features in accordance with one or more embodiments of the present invention is provided. The computer or computing device 100 of FIG. 1 contains an example of an environment for the execution of at least some of the computer code involved in performing the inventive methods, such as the block 150 of the computer-implemented method for differentiating patterns of care (DPOC) to detect anomalous subsets in any given population with a defined set of outcomes and features. In addition to the computer-implemented method for differentiating patterns of care (DPOC) to detect anomalous subsets in any given population with a defined set of outcomes and features of block 150, the computer or computing device 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and the computer-implemented method of block 1001, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

The computer 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of the computer-implemented method, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

The processor set 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In the computer-implemented method, at least some of the instructions for performing the inventive methods may be stored in the block 1001 of the computer-implemented method in persistent storage 113.

Communication fabric 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

Volatile memory 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

Persistent storage 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in the block 150 of the computer-implemented method typically includes at least some of the computer code involved in performing the inventive methods.

Peripheral device set 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

Network module 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

End user device (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

Remote server 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

Public cloud 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

Private cloud 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

Turning now to an overview of technologies that are more specifically relevant to aspects of the invention, variability in healthcare can be characterized by complex and diverse populations, interventions and outcomes. While scalable and unsupervised anomalous pattern detection methods can help distinguish whether outcomes and interventions among specific subpopulations are unusual, problems remain in terms of identifying and ranking anomalous subsets in populations, generalizing the identification of anomalous subsets for a large case of outcomes and expanding the analysis to multiple iterations in order to uncover different data insights.

For example, using conventional pattern detection methods, an analyst can correlate outcomes with some features to identify anomalies, one at a time or through an exhaustive grid search but, with tens to hundreds of features and values, analysis quickly becomes complicated and time consuming. While the analyst can filter the search, results can be biased by intuition or prior knowledge. Moreover, while the analyst may build a targeted clustering or prediction model to identify important features, such model may not reveal hidden anomalous patterns or help identify model biases.

Turning now to an overview of the aspects of the invention, one or more embodiments of the invention address shortcomings of the above-described approach by providing for a general data-driven process to identify and rank anomalous subsets in populations, to generalize identifications of anomalous subsets for large cases of outcomes and to expand analyses to multiple iterations in order to uncover different data insights. The data-driven process uses an unsupervised learning approach which automatically detects, ranks, summarizes and visualizes anomalous subgroups whereby actionable features and values can be emphasized through conditional scanning and iterations.

The above-described aspects of the invention address the shortcomings of known approaches by providing for a computer-implemented method for differentiating patterns of care (DPoC) to detect anomalous subsets in any given population with a defined set of outcomes and features. The computer-implemented method includes detecting the anomalous subsets, ranking the anomalous subsets based on a score of each anomalous subset that is reflective of an anomaly thereof, specifying whether each of the anomalous subsets overlaps with another one of the anomalous subsets, whether each of the anomalous subsets is unique and whether each of the anomalous subsets is conditional and specifying as to whether the detecting of each of the anomalous subsets has a higher or lower outcome than expected.

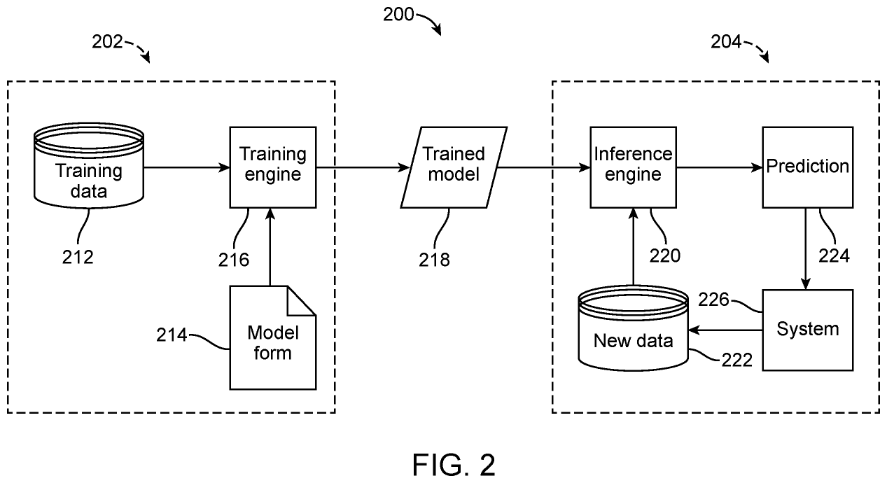

Turning now to a more detailed description of aspects of the present invention, FIG. 2 depicts a block diagram of components of a machine learning training and inference system 200. The machine learning training and inference system 200, in accordance with one or more embodiments of the invention, can utilize machine learning techniques to perform tasks, such as a computer-implemented method for differentiating patterns of care (DPOC) to detect anomalous subsets in any given population with a defined set of outcomes and features. In these or other cases, the computer-implemented method can include detecting the anomalous subsets, ranking the anomalous subsets based on a score of each anomalous subset that is reflective of an anomaly thereof, specifying whether each of the anomalous subsets overlaps with another one of the anomalous subsets, whether each of the anomalous subsets is unique and whether each of the anomalous subsets is conditional and specifying as to whether the detecting of each of the anomalous subsets has a higher or lower outcome than expected.

Embodiments of the invention utilize AI, which includes a variety of so-called machine learning technologies. The phrase “machine learning” broadly describes a function of electronic systems that learn from data. A machine learning system, engine, or module can include a trainable machine learning algorithm that can be trained, such as in an external cloud environment, to learn functional relationships between inputs and outputs, and the resulting model (sometimes referred to as a “trained neural network,” “trained model,” and/or “trained machine learning model”) can be used for managing information during a web conference, for example. In one or more embodiments of the invention, machine learning functionality can be implemented using an artificial neural network (ANN) having the capability to be trained to perform a function. In machine learning and cognitive science, ANNs are a family of statistical learning models inspired by the biological neural networks of animals, and in particular the brain. ANNs can be used to estimate or approximate systems and functions that depend on a large number of inputs. Convolutional neural networks (CNN) are a class of deep, feed-forward ANNs that are particularly useful at tasks such as, but not limited to analyzing visual imagery and natural language processing (NLP). Recurrent neural networks (RNN) are another class of deep, feed-forward ANNs and are particularly useful at tasks such as, but not limited to, unsegmented connected handwriting recognition and speech recognition. Other types of neural networks are also known and can be used in accordance with one or more embodiments of the invention described herein.

ANNs can be embodied as so-called “neuromorphic” systems of interconnected processor elements that act as simulated “neurons” and exchange “messages” between each other in the form of electronic signals. Similar to the so-called “plasticity” of synaptic neurotransmitter connections that carry messages between biological neurons, the connections in ANNs that carry electronic messages between simulated neurons are provided with numeric weights that correspond to the strength or weakness of a given connection. The weights can be adjusted and tuned based on experience, making ANNs adaptive to inputs and capable of learning. For example, an ANN for handwriting recognition is defined by a set of input neurons that can be activated by the pixels of an input image. After being weighted and transformed by a function determined by the network's designer, the activation of these input neurons are then passed to other downstream neurons, which are often referred to as “hidden” neurons. This process is repeated until an output neuron is activated. The activated output neuron determines which character was input. It should be appreciated that these same techniques can be applied in the case of localizing a target object referred by a compositional expression from an image set with similar visual elements as described herein.

The machine learning training and inference system 200 performs training 202 and inference 204. During training 202, a training engine 216 trains a model (e.g., the trained model 218) to perform a task. Inference 204 is the process of implementing the trained model 218 to perform the task in the context of a larger system (e.g., a system 226).

The training 202 begins with training data 212, which can be structured or unstructured data. The training engine 216 receives the training data 212 and a model form 214. The model form 214 represents a base model that is untrained. The model form 214 can have preset weights and biases, which can be adjusted during training. It should be appreciated that the model form 214 can be selected from many different model forms depending on the task to be performed. For example, where the training 202 is to train a model to perform image classification, the model form 214 can be a model form of a CNN (convolutional neural network). The training 202 can be supervised learning, semi-supervised learning, unsupervised learning, reinforcement learning, and/or the like, including combinations and/or multiples thereof. For example, supervised learning can be used to train a machine learning model to classify an object of interest in an image. To do this, the training data 212 includes labeled images, including images of the object of interest with associated labels (ground truth) and other images that do not include the object of interest with associated labels. In this example, the training engine 216 takes as input a training image from the training data 212, makes a prediction for classifying the image, and compares the prediction to the known label. The training engine 216 then adjusts weights and/or biases of the model based on results of the comparison, such as by using backpropagation. The training 202 can be performed multiple times (referred to as “epochs”) until a suitable model is trained (e.g., the trained model 218).

Once trained, the trained model 218 can be used to perform inference 204 to perform a task. The inference engine 220 applies the trained model 218 to new data 222 (e.g., real-world, non-training data). For example, if the trained model 218 is trained to classify images of a particular object, such as a chair, the new data 222 can be an image of a chair that was not part of the training data 212. In this way, the new data 222 represents data to which the model 218 has not been exposed. The inference engine 220 makes a prediction 224 (e.g., a classification of an object in an image of the new data 222) and passes the prediction 224 to the system 226. The system 226 can, based on the prediction 224, taken an action, perform an operation, perform an analysis, and/or the like, including combinations and/or multiples thereof. In some embodiments of the invention, the system 226 can add to and/or modify the new data 222 based on the prediction 224.

In accordance with one or more embodiments of the invention, the predictions 224 generated by the inference engine 220 are periodically monitored and verified to ensure that the inference engine 220 is operating as expected. Based on the verification, additional training 202 can occur using the trained model 218 as the starting point. The additional training 202 can include all or a subset of the original training data 212 and/or new training data 212. In accordance with one or more embodiments of the invention, the training 202 includes updating the trained model 218 to account for changes in expected input data.

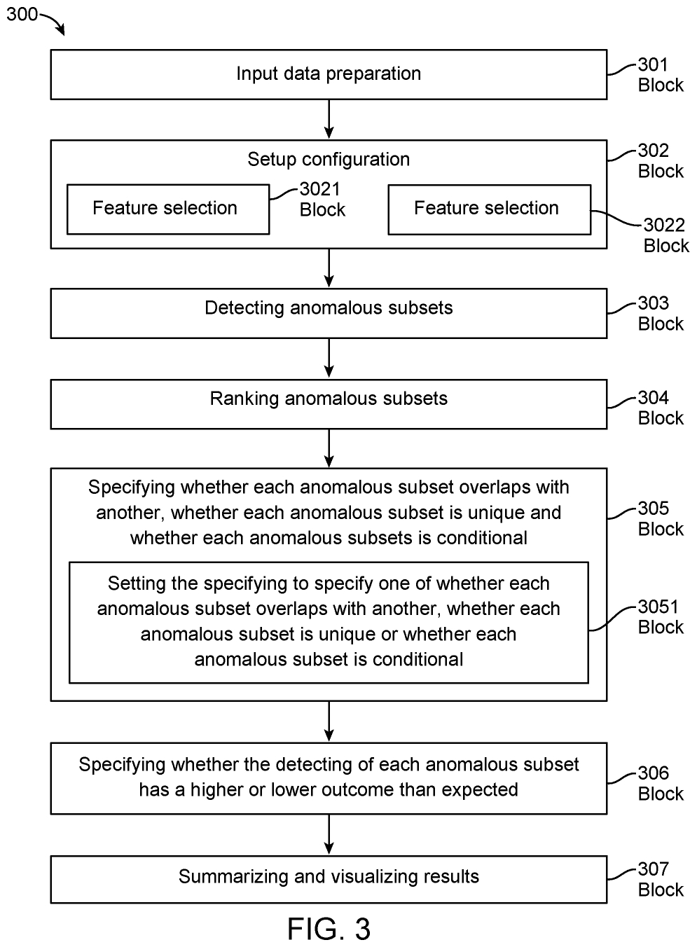

With reference to FIG. 3, a computer-implemented method 300 is provided for differentiating patterns of care (DPoC) to detect anomalous subsets in any given population with a defined set of outcomes and features. A shown in FIG. 3, the computer-implemented method 300 includes detecting the anomalous subsets (block 303), ranking the anomalous subsets based on a score of each anomalous subset that is reflective of an anomaly thereof (block 304), specifying whether each of the anomalous subsets overlaps with another one of the anomalous subsets, whether each of the anomalous subsets is unique and whether each of the anomalous subsets is conditional (block 305) and specifying as to whether the detecting of each of the anomalous subsets has a higher or lower outcome than expected (block 306). The computer-implemented method can further include summarizing and visualizing results (i.e., within reports) of the detecting of block 303, the ranking of block 304, the specifying of whether each of the anomalous subsets overlaps with another one of the anomalous subsets, whether each of the anomalous subsets is unique and whether each of the anomalous subsets is conditional of block 305 and the specifying as to whether the detecting of each of the anomalous subsets has a higher or lower outcome than expected of block 306 (block 307).

The computer-implemented method 300 is characterized in that input data preparation (block 301), setup configuration (block 302), algorithm executions of blocks 303-306 and report generations of block 307 are automated, with input data including binary or numerical outcome data and feature data with an unlimited number of features. In accordance with one or more embodiments of the present invention, the setup configuration of block 302 can include at least one or more of a feature selection operation in which the features of the unlimited number of features are selected (block 3021) and a parameter choice operation in which parameters include regularization parameters, counts for bootstrap repetitions, randomization initializations, numbers of the anomalous subsets to be identified and anomaly detection directions (block 3022). Also, in accordance with one or more embodiments of the present invention, the specifying of whether each of the anomalous subsets overlaps with another one of the anomalous subsets, whether each of the anomalous subsets is unique and whether each of the anomalous subsets is conditional of block 305 can include setting the specifying to specify one of whether each of the anomalous subsets overlaps with another one of the anomalous subsets, whether each of the anomalous subsets is unique or whether each of the anomalous subsets is conditional (block 3051).

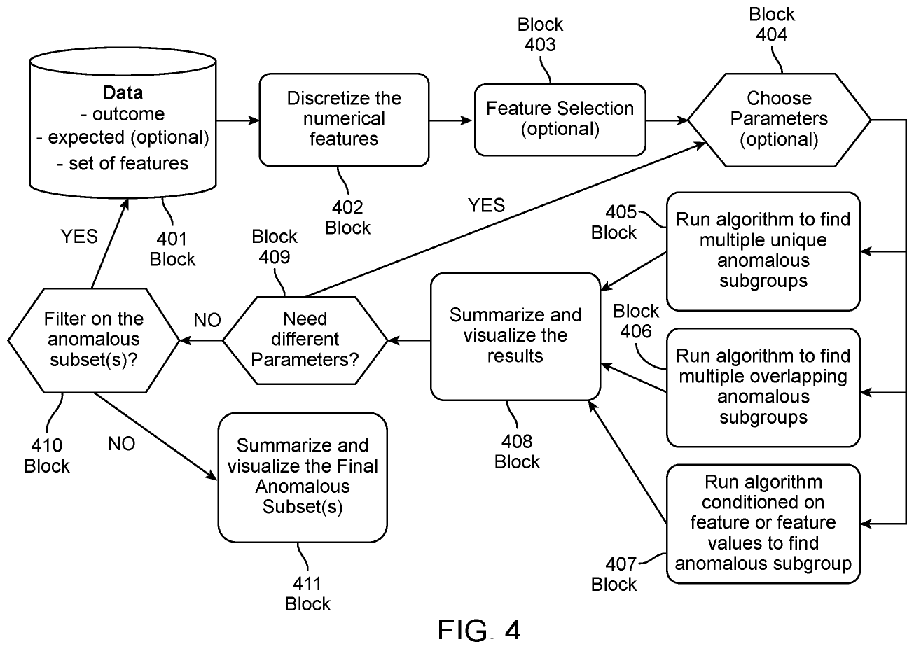

With reference to FIG. 4, the computer-implemented method 300 of FIG. 3 can be visualized further. As shown in FIG. 4, at an initial operation, data relating to outcomes, expected outcomes and sets of features are input in binary or numerical form (block 401). This data is then optionally discretized (block 402), feature selections are optionally made (block 403) and parameters are optionally chosen (block 404). Next, the algorithm(s) of the computer-implemented method 300 are run to detect anomalous subsets (block 405), to detect overlapping subsets (block 406) and to detect conditioning subsets (block 407). The results of the running of the algorithm(s) are then summarized and visualized (block 408) and determinations are made as to whether different parameter choices are needed (block 409), whereupon control reverts to block 404 if the determination is affirmative, and as to whether filtering is needed (block 410), whereupon control reverts to block 401 if the determination is affirmative. Subsequently, results are re-summarized and re-visualized (block 411).

The computer-implemented method 300 of FIG. 3 and the method of FIG. 4 can be executed in multiple and various settings, especially in healthcare settings. For example, the computer-implemented method 300 of FIG. 3 and the method of FIG. 4 can be used to identify subsets within Medicare Advantage populations using claims and social determinants of health data with anomalous quality measure compliance (e.g., medication adherence) and to inform outreach programs. As another example, the computer-implemented method 300 of FIG. 3 and the method of FIG. 4 can be used to identify factors driving higher surgery rates or complication rates in population subsets.

As a first exemplary use case, there is a need to identify a subset of a population with short term disability (STD) claims that have a higher percentage of switching into long term disability (LTD) benefits. In the case study population, there are 4.4% members who switch to LTD. The features considered in the case study are derived from healthcare usage, clinical conditions, enrollment information and job characteristics. Using the algorithm(s) and processes described above, it is possible to identify a subpopulation where the percentage of people switching to LTD is 18.5% with an odds ratio of 8.1.

As a second exemplary use case, there is a need to identify subsets from a population of people with major joint replacements with higher (hot spot) complication rates after adjusting for their risks based on comorbidities. In the case study population, the adjusted expected complication rate is 3%. The features considered in the case study are directly related to actions or choices that providers can take for possible interventions. They include the site of service, the length of stay, the day of the week when procedure is done and the type of discharge. The subpopulation identified from the algorithm(s) and processes described above indicates hot spot occurrences for inpatient surgeries with an extended length of more than four days of stay. The complication rate in the hot spot group is 10% with an odds ratio of 4.98.

As a third exemplary use case, there is a need to identify multiple subsets from a population of people who have higher rates of opioid usage through use of healthcare history and social determinants. In the case study population, the percentage of opioid users is 13% and identification of the subsets with higher percentages of opioid users was based on social-demographics, enrollment, healthcare use, health status and comorbidities. In this example, the algorithm(s) and processes described above identify the top 3 most anomalous nested subsets that are increasingly anomalous with higher percentages of opioid users but that decrease in size with odds ratios increasing from 4.6 to 10.5 and sizes decreasing from 10.9% to 0.4%.

In accordance with one or more embodiments of the present invention, executions of the algorithm(s) and processes described above search over all subsets to identify an optimal subset using various techniques. This is achieved using a scoring function analysis method, such as linear time subset scanning, to speed up searches along with enhancements to find targeted anomalous subsets efficiently and to provide for interpretations of results. These include, but are not limited to, outcome analysis and predictive bias assessment. Outcome analysis uses automated stratification (AS) to find subsets with the most differences between actual and population averages. Predictive bias assessment uses bias scanning to find subsets with the most differences between actual outcome and predicted probabilities and can be used to detect model biases.

AS generally focuses on identifying anomalous subsets of records that have significantly higher/lower-than-expected outcomes with a goal of maximizing a scoring function over all subsets to finds the subsets that have the highest scores, where the scoring function must satisfy a linear time subset scanning property that enables the search to be conducted in linear time.
Let
   D be the dataset
   X be the set of discretized features
   y∈{0,1} be the binary outcome
   \hat{p}ᵢ=y̅ be expected outcome for each record
For any given subset S, define score_{bias}(S) as a likelihood ratio score

$${H}_{0} : odds ( {y}_{i} ) = \frac{{\hat{p}}_{i}}{1 - {\hat{p}}_{i}} ∀ i ∈ D$$

$${H}_{1} : odds ( {y}_{i} ) = q \frac{{\hat{p}}_{i}}{1 - {\hat{p}}_{i}} , where q > 1 ∀ i ∈ S$$

$$\begin{matrix}{score}_{bias} ( S ) = {max}_{q} log {\prod }_{i ∈ S} \frac{Bernoulli ( \frac{q {\hat{p}}_{i}}{1 - {\hat{p}}_{i} + q {\hat{p}}_{i}} )}{Bernoulli ( {\hat{p}}_{i} )} \\ = {max}_{q} log ( q ) {\sum }_{i ∈ S} {y}_{i} - {\sum }_{i ∈ S} log ( 1 - {\hat{p}}_{i} + q {\hat{p}}_{i} )\end{matrix}$$

Maximize score_{bias}(S) over all subsets

$$S *= MDSS ( D , {\hat{p}}_{i} , {score}_{b i a s} )$$

Evaluating significance using parametric bootstrapping

Linear time subset scanning (LTSS) maximizes a scoring function f(s) over multiple subsets, where maximizing f(s) over all possible subsets has a search space of O(2^{N}). To scan in linear time, LTSS uses a priority function to sort feature values of a covariate and scanning is done over top k feature values, for k=1 . . . . N such that the highest-scoring subset is guaranteed to be one of the top k highest priority feature values, for k=1 . . . . N and all other subsets are suboptimal and need not be considered. Thus, the search space can be reduced from O(2^{N}) to O(N).

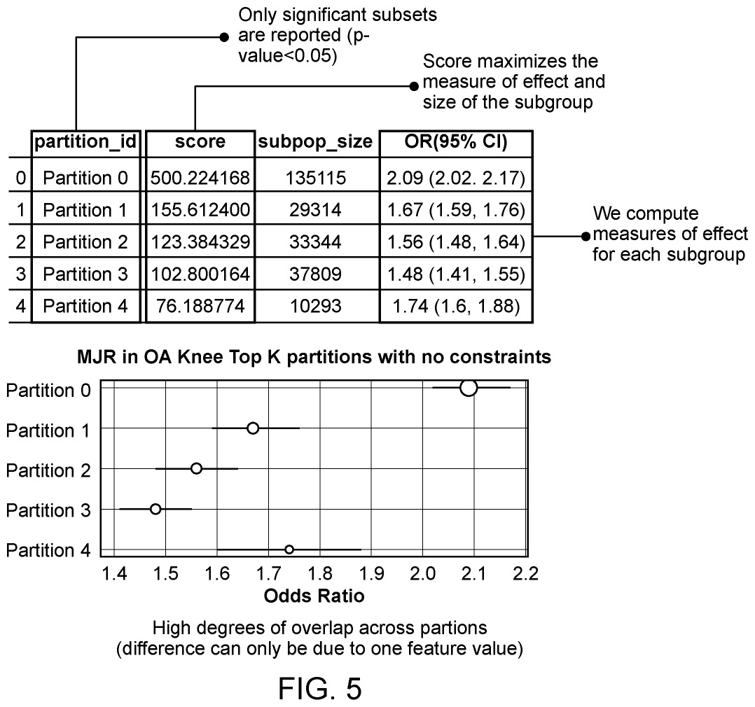

With reference to FIG. 5, for identifying and ranking subsets, there can be a goal of identifying two or more partitions when maximizing the scoring function. The identifying and ranking of subsets can include randomly ordering features to scan, scanning to identify a list of top K highest scoring subsets (local optimum), repeat the random ordering and the scanning N times (e.g., N=50) and identifying a final list of top K highest scoring subsets (global optimum).

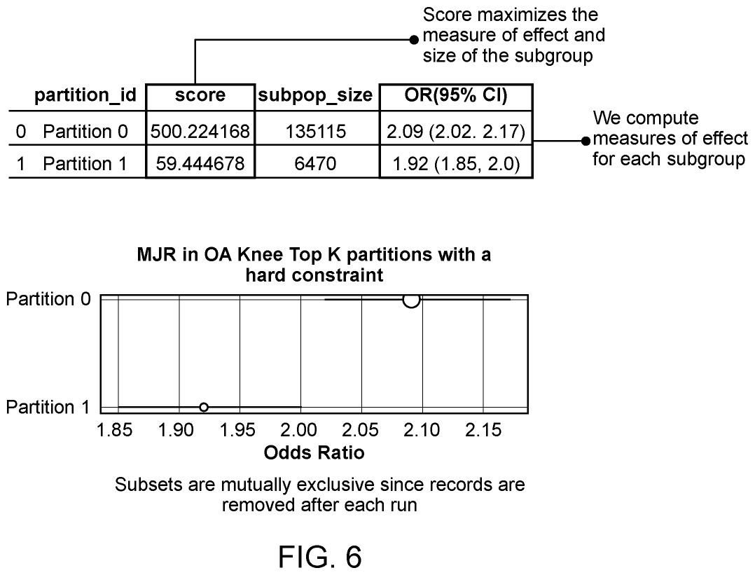

With reference to FIG. 6, for identifying and ranking mutually exclusive subsets, there can be a goal of recursively identifying two or more mutually exclusive subsets that show the most evidence of higher-than-expected outcomes. The identifying and ranking of mutually exclusive subsets can include identifying a highest scoring subset, removing records for the highest scoring subset and repeating the identifying and the removing K times.

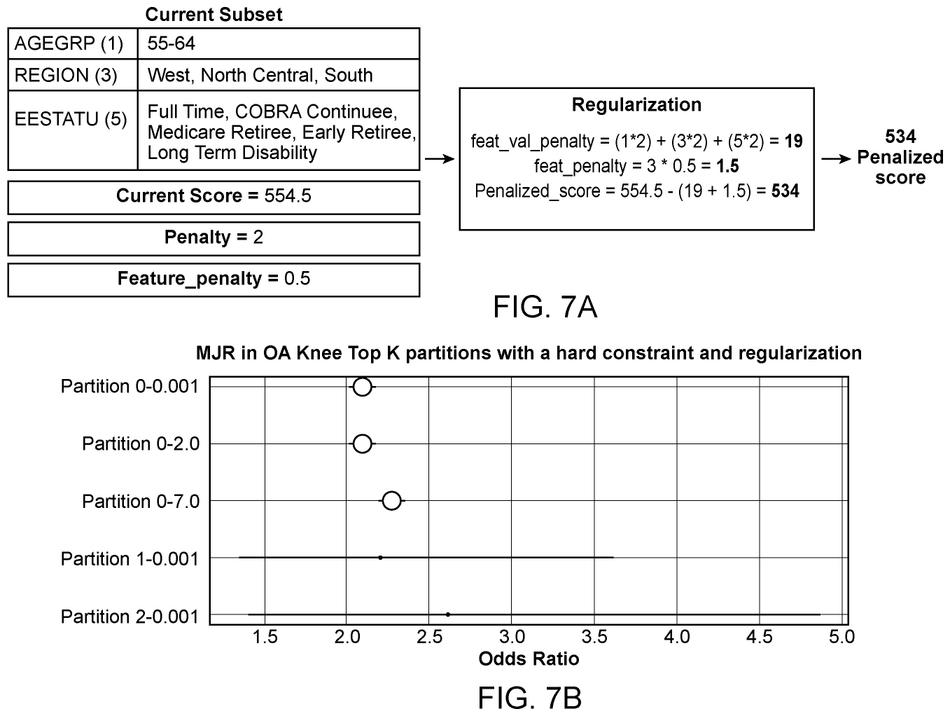

With reference to FIG. 7, for regularization to control complexity, there can be a goal to use regularization factors (penalties) to control a size of feature values and/or features in a subset. The regularization to control complexity can include identifying larger and less complex subsets, can be integrated and applied in detecting multiple subsets and can be used to control the identification of overlapping subsets.

Various embodiments of the invention are described herein with reference to the related drawings. Alternative embodiments of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” may be understood to include any integer number greater than or equal to one, i.e. one, two, three, four, etc. The terms “a plurality” may be understood to include any integer number greater than or equal to two, i.e. two, three, four, five, etc. The term “connection” may include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A computer-implemented method for differentiating patterns of care (DPoC) to detect anomalous subsets in any given population with a defined set of outcomes and features, the computer-implemented method comprising:
detecting the anomalous subsets;
ranking the anomalous subsets based on a score of each anomalous subset that is reflective of an anomaly thereof;
specifying whether each of the anomalous subsets overlaps with another one of the anomalous subsets, whether each of the anomalous subsets is unique and whether each of the anomalous subsets is conditional; and
specifying as to whether the detecting of each of the anomalous subsets has a higher or lower outcome than expected.

2. The computer-implemented method according to claim 1, wherein the computer-implemented method is characterized in that input data preparation, setup configuration, algorithm executions and report generations are automated.

3. The computer-implemented method according to claim 2, wherein the input data comprises binary or numerical outcome data and feature data with an unlimited number of features.

4. The computer-implemented method according to claim 3, further comprising a feature selection operation in which the features of the unlimited number of features are selected.

5. The computer-implemented method according to claim 3, further comprising a parameter choice operation in which parameters comprise regularization parameters, counts for bootstrap repetitions, randomization initializations, numbers of the anomalous subsets to be identified and anomaly detection directions.

6. The computer-implemented method according to claim 1, wherein the specifying of whether each of the anomalous subsets overlaps with another one of the anomalous subsets, whether each of the anomalous subsets is unique and whether each of the anomalous subsets is conditional comprises setting the specifying to specify one of whether each of the anomalous subsets overlaps with another one of the anomalous subsets, whether each of the anomalous subsets is unique or whether each of the anomalous subsets is conditional.

7. The computer-implemented method according to claim 1, further comprising summarizing and visualizing results of the detecting, the ranking, the specifying of whether each of the anomalous subsets overlaps with another one of the anomalous subsets, whether each of the anomalous subsets is unique and whether each of the anomalous subsets is conditional and the specifying as to whether the detecting of each of the anomalous subsets has a higher or lower outcome than expected.

8. A computer program product for differentiating patterns of care (DPoC) to detect anomalous subsets in any given population with a defined set of outcomes and features, the computer program product comprising one or more computer readable storage media having computer readable program code collectively stored on the one or more computer readable storage media, the computer readable program code being executed by a processor of a computer system to cause the computer system to perform a method comprising:
detecting the anomalous subsets;
ranking the anomalous subsets based on a score of each anomalous subset that is reflective of an anomaly thereof;
specifying whether each of the anomalous subsets overlaps with another one of the anomalous subsets, whether each of the anomalous subsets is unique and whether each of the anomalous subsets is conditional; and
specifying as to whether the detecting of each of the anomalous subsets has a higher or lower outcome than expected.

9. The computer program product according to claim 8, wherein the method is characterized in that input data preparation, setup configuration, algorithm executions and report generations are automated.

10. The computer program product according to claim 9, wherein the input data comprises binary or numerical outcome data and feature data with an unlimited number of features.

11. The computer program product according to claim 10, wherein the method further comprises a feature selection operation in which the features of the unlimited number of features are selected.

12. The computer program product according to claim 10, wherein the method further comprises a parameter choice operation in which parameters comprise regularization parameters, counts for bootstrap repetitions, randomization initializations, numbers of the anomalous subsets to be identified and anomaly detection directions.

13. The computer program product according to claim 8, wherein the specifying of whether each of the anomalous subsets overlaps with another one of the anomalous subsets, whether each of the anomalous subsets is unique and whether each of the anomalous subsets is conditional comprises setting the specifying to specify one of whether each of the anomalous subsets overlaps with another one of the anomalous subsets, whether each of the anomalous subsets is unique or whether each of the anomalous subsets is conditional.

14. The computer program product according to claim 8, wherein the method further comprises summarizing and visualizing results of the detecting, the ranking, the specifying of whether each of the anomalous subsets overlaps with another one of the anomalous subsets, whether each of the anomalous subsets is unique and whether each of the anomalous subsets is conditional and the specifying as to whether the detecting of each of the anomalous subsets has a higher or lower outcome than expected.

15. A computing system comprising:
a processor;
a memory coupled to the processor; and
one or more computer readable storage media coupled to the processor, the one or more computer readable storage media collectively containing instructions that are executed by the processor via the memory to implement a method for differentiating patterns of care (DPoC) to detect anomalous subsets in any given population with a defined set of outcomes and features comprising:
detecting the anomalous subsets;
ranking the anomalous subsets based on a score of each anomalous subset that is reflective of an anomaly thereof;
specifying whether each of the anomalous subsets overlaps with another one of the anomalous subsets, whether each of the anomalous subsets is unique and whether each of the anomalous subsets is conditional; and
specifying as to whether the detecting of each of the anomalous subsets has a higher or lower outcome than expected.

16. The computing system according to claim 15, wherein the differentiating of patterns of care (DPoC) to detect anomalous subsets in any given population with the defined set of outcomes and features is characterized in that input data preparation, setup configuration, algorithm executions and report generations are automated.

17. The computing system according to claim 16, wherein:
the input data comprises binary or numerical outcome data and feature data with an unlimited number of features, and
the instructions further comprise a feature selection operation in which the features of the unlimited number of features are selected.

18. The computing system according to claim 16, wherein:
the input data comprises binary or numerical outcome data and feature data with an unlimited number of features, and
the instructions further comprise a parameter choice operation in which parameters comprise regularization parameters, counts for bootstrap repetitions, randomization initializations, numbers of the anomalous subsets to be identified and anomaly detection directions.

19. The computing system according to claim 15, wherein the specifying of whether each of the anomalous subsets overlaps with another one of the anomalous subsets, whether each of the anomalous subsets is unique and whether each of the anomalous subsets is conditional comprises setting the specifying to specify one of whether each of the anomalous subsets overlaps with another one of the anomalous subsets, whether each of the anomalous subsets is unique or whether each of the anomalous subsets is conditional.

20. The computing system according to claim 15, wherein the instructions further comprise summarizing and visualizing results of the detecting, the ranking, the specifying of whether each of the anomalous subsets overlaps with another one of the anomalous subsets, whether each of the anomalous subsets is unique and whether each of the anomalous subsets is conditional and the specifying as to whether the detecting of each of the anomalous subsets has a higher or lower outcome than expected.
